(19) United States
(12) Patent Application Publication
(10) Pub. No.: US 2023/0101729 A1
(43) Pub. Date: Mar. 30, 2023

(54) COMPOSITION AND METHOD FOR MODULATING HYDROGEN ION PHYSIOLOGY RELATED TO BRAIN HEALTH COMPONENT

(71) Applicant: Cannogen Biosciences Inc., Incline Village, NV (US)

(72) Inventor: Edward Larry McCLeary, Incline Village, NV (US)

(73) Assignee: Cannogen Biosciences Inc., Incline Village, NV (US)

(21) Appl. No.: 17/954,319

(22) Filed: Sep. 27, 2022

Related U.S. Application Data
(60) Provisional application No. 63/249,979, filed on Sep. 29, 2021.

(51) Int. Cl. A61K 31/05; A61K 31/16; A61K 31/015; A61K 31/7048; A61K 31/09; A61K 31/145; A61K 31/192; A61K 31/366; A61K 31/01; A61K 31/519; A61K 31/197; A61K 31/685; A61K 31/14; A61K 31/7076; A61K 31/385; A61K 31/122; A61K 31/198; A61K 31/19

(52) U.S. Cl. CPC A61K 31/05; A61K 31/16; A61K 31/015; A61K 31/7048; A61K 31/09; A61K 31/145; A61K 31/192; A61K 31/366; A61K 31/01; A61K 31/519; A61K 31/197; A61K 31/685; A61K 31/14; A61K 31/7076; A61K 31/385; A61K 31/122; A61K 31/198; A61K 31/19

(57) ABSTRACT

A nutritional supplement composition that extends the utility of the prior art methods to include enhanced utility for bone health via modulation of the RANK, RANKL, NFKB signaling system and incorporates unrelated, unforeseen benefits on brain health based on the beneficial impact of the same signaling system on brain health thus preventing loss of neurological function and/or normalizing impaired or deteriorating neurological function in humans based on: (1) mitigation of the adverse impact of dysregulated acid-base balance, (2) dysregulation of prostaglandin physiology, (3) abnormalities in RANK, RANKL, NFKB physiology. reversing elevated NFKB, RANK, RANKL signaling. The nutrients that are ideal for the desired result are Honokiol, Magnolol, Beta-caryophyllene, Palmitoylethanolamide, Hesperidin, Schisandrin, Ferulic Acid, Beta-carotene, Lutein, Lycopene, Sulforaphane, and Ellagic Acid.

### CROSS-REFERENCE TO RELATED APPLICATION

This non-provisional application relies for priority on U.S. Provisional Application No. 63/249,979, entitled “Composition and Method for Modulating Hydrogen Ion Physiology Related to Brain Health Component,” filed on 19 Sep. 2021, the entire specification of which is incorporated by reference as if fully set forth herein.

### BACKGROUND OF THE INVENTION

### 1. Field of the Invention

This invention relates in general to nutraceuticals, including functional foods, dietary supplements, nutritional supplements, medical foods, botanical drugs, and drugs and more particularly to nutraceuticals and methods that are useful for supporting and promoting a healthy calcium and muscle physiology and brain health.

### 2. Background Art

Currently it is estimated that approximately 10 million individuals have osteoporosis and another 34 million suffer from low bone density. If the effectiveness of prevention efforts does not improve, it is estimated that the public health cost of osteoporosis, including hospitalizations due to broken bones, will rise to $200 billion per year by 2040.

Poor brain health is an epidemic. The brain begins showing signs of cognitive decline in people as early as their 20's. 3 in 5 persons will develop brain disease in their lifetime. By 2030, the total cost of Alzheimer's disease, dementia, and stroke alone is expected to exceed $1 trillion!

The majority of current treatments for osteoporosis are pharmaceutically based, such as Fosamax® and the statin category of drugs, or involves simple calcium or calcium/magnesium (Cal/Mag) supplementation. Despite aggressive therapy, many patients do not respond or improve. Most therapies, even if effective, are limited by side effects. These therapies may be associated with enhanced urinary calcium loss and increased calcium-based urinary stone formation. Even if a drug protocol is effective and without adverse effects for some sub-set of patients, it is required that a synthetic drug be taken every week for drugs such as Fosamax®, or every day for other drugs, for the rest of a patient's life—which could be three or four decades.

Muscle wasting also is on the rise in our society. It long has been associated with increasing age, though now it is recognized as a problem that begins much earlier. The therapy for this, up to now, has been limited to increasing exercise, increasing food intake, focusing on protein, minerals and vitamins; that is essentially maintaining a healthy diet in older people.

There is no effective treatment for most brain disorders, even high profile ones such as Alzheimer's disease. There are pharmaceuticals that have been cleared for use by the FDA. However, not a single one can come close to curing the disorder. At best, symptoms improve for about 6 months under optimal circumstances. Two leading theories why this is the case are (1) that the diagnosis is made too late at a time when nerve cells are dead or are beyond a state where intervention can make a meaningful difference, or (2) that drugs impact only one contributory of several pathway responses for the disease or disorder.

Therefore, combined nutritional approaches, which traditionally are associated with a much diminished profile of adverse reactions, are currently being explored as optional approaches because they generally can be formulated to beneficially impact many of the disease causing mechanisms.

The same is true of bone disorders such as osteoporosis, which is multi-factorial in nature. Diet-induced chronic metabolic acidosis which may be responsible for degeneration of bone tissue (such as skeletal and dental/jaw) includes two basic mechanisms: (1) physico-chemical dissolution at the bone surface, ultimately resulting in bone loss, and (2) activation of osteoclast activity. Osteoclasts are cells that break down bone. Upregulation of prostaglandin signaling by turning on various COX enzymes (cyclo-oxygenase) that further enhances bone loss are believed to be major contributing factors. A nutritional approach that includes ingestion of acid buffering nutrients in combination with botanical COX inhibiting nutrients are able to reverse the adverse impact of chronic diet-induced metabolic acidosis (usually with the serum pH remaining in the normal region but the serum bicarbonate level decreasing) on such bone tissues.

Prior attempts to treat these conditions with dietary and food supplements has been an ongoing endeavor, both by the present inventor and others. One highly effective method of treatment has been introduced by the inventor of the present invention in U.S. Pat. No. 6,964,969 entitled “Composition and Method for Treating Impaired or Deteriorating Neurological Function” to McCleary and U.S. Pat. No. 8,703,209 entitled “Composition and Method for Modulating Hydrogen Ion Physiology,” also to McCleary, the disclosures of which are both incorporated by reference as if fully set forth herein.

As a result of recent advances on the field, new information has come to light that includes additional methods of achieving improved results in combatting bone deterioration and muscle mass loss. The purpose of this application is to extend the coverage of the existent Hydrogen ion physiology (HIP) regime, and to incorporate novel supplements and food additives related to the bone and muscle mass, as well as brain health components.

### SUMMARY OF THE INVENTION

A nutritional supplement composition for improving disordered bone metabolism and normalizing impaired or deteriorating neurological function in humans is composed of: interventions that address the underlying mechanisms for both groups of disorders. It is counterintuitive that a novel set of metabolic signaling pathways can address common underlying mechanisms involving both bone and brain deterioration. The unexpected link seems to be excessive activity of the RANKL (Receptor Activator of Nuclear Factor Kappa B Ligand), RANK (receptor Activator of Nuclear Factor Kappa B), NFKB (Nuclear Factor Kappa B) and related downstream pathways. Acting together, they work in tandem to produce (1) physico-chemical dissolution at the bone surface, ultimately resulting in bone loss, and (2) activation of osteoclast activity via a cell-mediated biological mechanism that produces microscopic breakdown of bone, (3) also damage to nerve cells via a number of mechanisms including (4) excessive inflammation in the nervous system due to both the release of inflammatory chemicals called cytokines and the activation of inflammatory immune cells called microglia, (5) activation of neuronal cell death pathways, (6) generation of oxidative free radical generation leading to damage of vital cellular structures including cell membranes, genetic material and enzymes that control metabolic pathways, and (7) physiological abnormalities of critical brain communication compounds called neurotransmitters (such as, for example, acetylcholine, a neurotransmitter essential for memory function).

In accordance with the present invention, there is described and claimed a composition for supporting and promoting healthy hydrogen ion balance, healthy calcium metabolism, healthy muscle metabolism, and for optimizing and preventing deterioration of brain health in the body of a human being, said composition comprising the following nutrients: a first component selected from the group consisting of Honokiol, Magnolol, Palmitoylethanolamide and Beta-Caryophyllene, each of which nutrients beneficially modulate the endocannabinoid metabolism to diminish the adverse impact of stress on neural tissues, a second component selected from the group consisting of Hesperidin, Schisandrin and Sulforaphane, each of which nutrients beneficially modulate a variety of cellular and molecular pathways of brain inflammation, and a third component selected from the group consisting of Ferulic Acid, Ellagic Acid, Lutein, Beta-Carotene and Lycopene, each of which nutrients beneficially act via different pathways to mitigate the development of amyloid beta neurodegenerative pathology, the first, second, and third components collectively provided in an effective amount for supporting and promoting healthy hydrogen ion balance, healthy calcium metabolism, and healthy muscle metabolism in the body of a human being.

Also disclosed and claimed is a method of administering to a human or other being, an effective amount of the above described composition, comprising the steps of 1) formulating the above described composition and 2) administering the composition by oral intake, absorption, circulatory transport or interstitial flux.

### DETAILED DESCRIPTION OF THE PREFERRED EMBODIMENTS

It has been recently discovered that the RANKL/NFKB signaling pathway plays a critical role in osteoclast activation and bone loss. It has also been shown that the acidosis/COX pathway (as taught in the current HIP patent) activates the RANKL/NFKB signaling pathway. Further, it has been shown that the acidosis/COX pathway and the RANK/RANKL/NFKB pathways converge and act together to turn on osteoclast activity resulting in markedly accelerated bone loss.

One novel aspect of the present invention is that while the acidosis/COX pathway and the RANKL/NFKB pathway independently up-regulate osteoclast activity, they further enhance each other's biological impact by feeding into the same downstream signaling system. Thus, by combining novel nutritional approaches that beneficially impact multiple pathways and mechanisms of disease, synergistic benefits accrue and enhanced and more effective interventions are available.

Nutrients have been discovered that are suitably positioned to inhibit, or block the RANK, the RANKL and the NFKB signal pathways, thus markedly enhancing the effect of the prior known methods because of the impact on convergent pathways and the subsequently markedly enhanced benefits resulting from the synergistic effect of addressing several debilitating processes by adding multiple novel ways to reduce their detrimental effect.

Additional synergistic enhancements of the new novel nutrients that reduce signaling in the RANK/RANKL/NFKB pathways, also produce an array of beneficial unexpected “downstream” metabolic effects involving multiple different additional biochemical pathways such as cannabinoid, opiate, PPAR alpha, GPR55, and additional epigenetic signaling systems, thus extending the aggregate benefits beyond those initially anticipated. This means that by beneficially modulating these additional, unrelated pathways, the combined benefits far exceed the beneficial effects of any two individually applied approaches.

In addition, chronic metabolic acidosis has been shown to impair insulin-signaling in a manner that results in a biochemical and hormonal situation that is referred to as “insulin resistance.” Insulin resistance plays a major role in the development of diabetes, vascular disease, cardiovascular disease (by its impact on lipid metabolism (such as producing deleterious impact on serum cholesterol and triglyceride levels, for example)) and neurodegeneration. It has been shown that cerebral insulin resistance is a contributory factor to neurodegeneration and neuroinflammation, both of which are present in disorders such as Alzheimer's disease, vascular dementia, other dementing diseases, brain or other central nervous system injury, hemorrhage, stroke and so forth. Upregulation of RANK/RANKL/NFKB signaling plays key roles in these disorders. Down-regulation of this signaling cascade has been shown to ameliorate the underlying contributory factors and provide symptomatic improvement. The additional nutrients are specifically noted to down-regulate the RANK/RANKL/NFKB signal pathways in bone and brain. In addition, because of their beneficial impact on a variety of additional related metabolic pathways (as noted above) relying on unrelated and unexpected mechanisms, heretofore unforeseen neurochemical and neurobehavioral benefits accrue.

Additional novel approaches that synergize with the known procedures, for example, those described in aforementioned U.S. Pat. Nos. 6,964,969 and 8,703,209, activate effects on bone health (by their beneficial impact on RANK/RANKL/NFKB signaling) and provide a novel approach, in conjunction with the previously taught nutrients), that unexpectedly enhances brain function and prevents/preserves loss of nervous system function.

The nutrients taught in prior art patents referenced above that beneficially modulate the RANK/RANKL/NFKB pathway include: folic acid, trimethylglycine, phosphatidyl choline, L-camitine, acetyl L-camitine, B vitamins, glyceryl phosphorylcholine, choline, SAMe (S adenosylmethionine), creatine, lipoic acid, coenzyme Q10, L-aspartic acid, serine, glycine and pyruvate.

Additional agents which augment bone and/or muscle metabolism by various unrelated or not well-understood mechanisms include vitamin D (preferably vitamin D3), vitamin K, vitamin C, vitamin B5, boron, ipriflavone or other isoflavones, and branched chain amino acids (BCAA)

Novel nutrients that are combinable in various formulations dictated by the specific modulation of the desired signaling systems that are required are proposed. These novel nutrients may be used in combination with each other, or in further commination with the above listed nutrients disclosed in aforementioned U.S. Pat. Nos. 6,964,969 and 8,703,209. The novel nutrients include, inter alia: Honokiol, Magnolol, Beta-caryophyllene, Palmitoylethanolamide, Hesperidin, Schisandrin, Ferulic Acid, Beta-carotene, Lutein, Lycopene, Sulforaphane, and Ellagic Acid.

The invention herein has been described with reference to the disclosed embodiments including the combinations and permutations of the several nutrients, but it should be understood that the features and operation of the invention as described is susceptible to modification or alteration without departing significantly from the spirit of the invention. For example, the types and effective amounts of the various nutrients may be changed and or may be altered to fit specific applications. Additional similar nutrients may be uncovered that provide for addressing similar pathways that enhance bone health and provide for brain health. Accordingly, the specific embodiments described herein are for illustrative purposes only and the invention is not limited except by the following claims.

What is claimed is:

1. A composition for supporting and promoting healthy hydrogen ion balance, healthy calcium metabolism, healthy muscle metabolism, and optimizing and preventing deterioration of brain health in the body of a human being, said composition comprising the following nutrients: a first component selected from the group consisting of Honokiol, Magnolol, Palmitoylethanolamide and Beta-Caryophyllene, each of which nutrients beneficially modulate the endocannabinoid metabolism to diminish the adverse impact of stress on neural tissues; a second component selected from the group consisting of Hesperidin, Schisandrin and Sulforaphane, each of which nutrients beneficially modulate a variety of cellular and molecular pathways of brain inflammation; and a third component selected from the group consisting of Ferulic Acid, Ellagic Acid, Lutein, Beta-Carotene and Lycopene each of which nutrients beneficially act via different pathways to mitigate the development of amyloid beta neurodegenerative pathology; said first, second, and third components collectively provided in an effective amount for supporting and promoting healthy hydrogen ion balance, healthy calcium metabolism, and healthy muscle metabolism in the body of said human being.

2. A composition as in claim 1 wherein at least one of said first, second and third components include at least one ingredient selected from the group consisting of folic acid, trimethylglycine, phosphatidyl choline, L-camitine, acetyl L-camitine, B vitamins, glyceryl phosphorylcholine, choline, SAMe (S adenosylmethionine), creatine, lipoic acid, coenzyme Q10, L-aspartic acid, serine, glycine and pyruvate.

3. A method of administering an effective amount of the composition of claim 1, comprising the steps of:
1) formulating the composition and
2) administering the composition by oral intake, absorption, circulatory transport and interstitial flux.
